(19) United States
(12) Patent Application Publication
(10) Pub. No.: US 2010/0056901 A1
(43) Pub. Date: Mar. 4, 2010

(54) APPARATUS AND METHOD FOR MAGNETIC RESONANCE SCANNING

(75) Inventor: Christopher Paul Randell, Wotton-under-Edge (GB)

Correspondence Address: OLIFF & BERRIDGE, PLC, P.O. BOX 320850, ALEXANDRIA, VA, 22320-4850, US

(73) Assignee: PULSETEQ LIMITED, WOTTON-UNDER-EDGE (GB)

(21) Appl. No.: 12/450,769

(22) PCT Filed: May 9, 2008
(86) PCT No.: PCT/GB2008/001606

§ 371 Date: Oct. 13, 2009

(30) Foreign Application Priority Data
May 11, 2007 (GB) 0709114.3

(51) Int. Cl. A61B 5/055

(52) U.S. Cl. 600/414; 600/422

(57) ABSTRACT

An apparatus and method for magnetic resonance scanning is described. The apparatus comprises an RF coil (16) for receiving an RF signal, a housing (10) for housing the RF coil, and a support (38). The support (38) is provided for locating a fiducial marker (50) in a repeatable position relative to the housing (10), the repeatable position being within the RF field of the RF coil (16). The fiducial marker, in use, provides a reference peak in a magnetic resonance spectroscopy spectrum. Methods of using such apparatus for magnetic resonance scanning, are also outlined. In particular a method is described that includes the step of providing a housing (10) that comprises a recess (38) located within the RF field of the RF coil (16). A fiducial marker (50) is then placed in the recess without exposing the RF coil (16).

The present invention relates to an apparatus and method for magnetic resonance scanning. In particular the present invention relates to apparatus for magnetic resonance spectroscopy comprising at least one fiducial marker.

Magnetic resonance spectroscopy (MRS) is known and involves placing various chemical compounds inside a strong magnetic field and using radio wave frequencies to excite atoms in these compounds. Some atoms, such as Hydrogen and Phosphorus, absorb and emit radio energy at a range of frequencies which are characteristic for that atom in a particular chemical compound. Peaks in the spectra that occur at such characteristic frequencies can be used to identify the presence of a variety of alternative chemical compounds. One known form of MRS is chemical shift imaging.

MRS can be performed using various types of magnetic resonance apparatus to scan areas of a human body to identify compounds such as Glycogen, lipids, phosphates, ATP (Adenosine Triphosphate), and many more. For example, Magnetic resonance imaging apparatus may be used for chemical shift imaging. The range of compounds that can be identified in the investigation depend on the atom which is excited.

Magnetic resonance (MR) apparatus for performing MRS typically comprises a large magnet for generating a static magnetic field, one or more RF coils for transmitting RF pulses to perturb the magnetic field and one or more RF receive coils for monitoring the effect of the perturbation to extract MR information. It is known to provide one of the RF receive coils in a housing that can be positioned over an area of interest of a subject prior to the subject being moved into the MR scanner. Information gathered by this RF receive coil can then be analysed by a spectrometer to provide a plot of signal intensity versus excitation frequency for CONFIRMATION COPY the area of interest, thereby providing information on the presence and concentration of chemical compounds in that area.

One way to calculate the quantity of each identified compound that is present is to provide a reference peak in the spectrum. Fiducial markers (a known amount of a particular compound) can be used to provide such reference peaks. Quantities of identified chemical compounds can be calculated relative to the quantity of fiducial material present.

It is known to place a glass vial containing a fiducial marker on the surface of the area to be scanned on the subject, before the RF coil is positioned over the same area, and before the subject is moved into the scanner. This has the disadvantage that the position of the fiducial with respect to the RF coil varies from experiment to experiment. The position of the fiducial with respect to the RF coil is not easily repeatable and therefore results from one experiment to the next may not be repeatable. This may, for example, make it hard to compare results from various experiments over time.

Placing a glass vial containing a fiducial marker on the surface of the area to be scanned, as described above, can also prevent the RF coil from getting as close to the surface of the area being scanned as would be possible without the fiducial marker. If the fiducial marker increases the distance between the RF coil and the surface of the area being scanned, the result can be a decrease in signal strength leading to a decrease in signal to noise ratio in the spectra, thereby decreasing the resolution of the spectral analysis. Additionally, placing the fiducial on the skin of the patient can compromise the comfort of the procedure.

Alternatively, it is known to glue or tape glass vials containing fiducial markers wherever they will fit on the inside of the housing containing the RF coils. When the housing is sealed, to prevent the RF coils from being accessed, the fiducials are rendered inaccessible. The only way to access the fiducial is to break open the housing, thus exposing the RF coils. This is unacceptable as part of a normal use of an RF coil and could lead to safety issues. The RF coils carry high electric voltage, exposing them can be extremely dangerous for the user, and is thus highly undesirable. Additionally, this method also has the disadvantage that the position of the fiducial with respect to the RF coil is not easily repeatable as described previously.

Other types of fiducial markers, such as positional fiducial markers, are also known. These fiducial markers comprise a substance located at a known position; they provide a reference position from which to calculate the position of other substances. For example, EP 1 356 773 describes a biopsy device which comprises an accurately placed fiducial marker, with reference to which the desired biopsy site location is stereotactically determined. Also, WO 2006/134357 describes an RF coil assembly for use in magnetic resonance imaging, the RF coil comprising a fiducial marker unit. The fiducial markers are positioned such that they act as a clear reference indicator in each image taken of the brain. The absolute and relative positions of the fiducial markers are known such that the position of other substances in the brain can be calculated relatively. Other examples of positional fiducial marker are described in US 2003/0220559 and JP 2002-306444.

According to a first aspect of the present invention, RF coil apparatus for magnetic resonance spectroscopy comprises:
at least one RF coil for receiving an RF signal; and
a housing for housing said at least one RF coil;
characterised in that a support is provided for locating a fiducial marker in a repeatable position relative to the housing, the fiducial marker providing a reference peak in a magnetic resonance spectroscopy spectrum, wherein said repeatable position is located within the RF field of said at least one RF coil.

The present invention thus provides an RF coil apparatus, with a support for locating a fiducial marker in a repeatable position relative to the housing, for use in magnetic resonance spectroscopy. For example, a fiducial marker comprising ‘material x’ could be held by the support in a fixed position and used in combination with the apparatus for a number of purposes. These include, but are not limited to, checking the shimming of the magnetic field, providing a reference signal at a specific frequency and measuring the concentration or quantity of ‘material x’ in a region of the body. These would all be achieved through either the resulting chemical shift spectrum or through chemical shift imaging. The use of a fiducial marker for providing a reference peak in an MRS spectra can thus be seen to differ from providing a positional fiducial marker of the type described above.

The present invention offers several advantages over the prior art described above. In particular it allows a fiducial, or a number of different fiducials, to be repeatably positioned with respect to the housing and thus with respect to the RF coil, giving continuity between experiments. Additionally, the structure results in a more comfortable experience for the patient being scanned.

Advantageously, the support enables a fiducial marker to be located in said repeatable position without exposing the RF coil. This enables the user to remove and replace the fiducial marker from the apparatus without exposing the RF coil. Exposing the RF coil would render the apparatus electrically unsafe, as there would no longer be a barrier between the user and the high electrical current running through the coil. The present invention thus allows a fiducial marker to be used in combination with an RF coil without the need to open the housing containing the coil. The present invention thus provides flexibility in the choice of chemical enclosed in the fiducial from scan to scan or study to study without compromising safety.

The housing may provide the support. For example the support may comprise a recess in the housing for receiving a fiducial marker. This has the advantage that it does not prevent the RF coil from getting close to the surface of the area of the subject to be scanned.

The support may alternatively comprise an attachment device for releasably retaining a fiducial marker unit. The fiducial marker unit may comprise a recess for receiving a fiducial marker. Preferably, the attachment device comprises a clip, screw, pressure fit, or bayonet structure. The fiducial marker unit may be provided between the RF coil and the subject, or alternatively, it may be provided in another location.

The recess may contain the fiducial substance. Alternatively the recess may receive a container containing the fiducial substance; in this case the recess retains the container in a fixed location using, for example a bung, clip, screw threaded, or bayonet type fitting. The recess may be suitable for both of the above uses, increasing the flexibility of the apparatus.

Preferably the recess has a substantially smooth profile. In this manner sharp edges are avoided in order to minimize variation in susceptibilities between particles of the fiducial substance.

Conveniently a lid is provided to seal the recess; this lid may be attachable to the housing or to the fiducial marker unit. Preferably this lid does not protrude substantially from the housing or the unit. This ensures that the RF coil can lie as close as possible to the surface of the area to be scanned. This is an advantage over prior art apparatus as the presence of a fiducial does not significantly increase the gap between the coil and the surface of the area to be scanned.

The recess may comprises a through hole into which a container containing the fiducial fits. For example, where the support comprises a recess, the recess may comprise a hole that extends through the centre of the RF coil.

The housing, the support, and/or the fiducial marker unit, is preferably constructed from plastics, or any other material that has no affect on either the RF or magnetic fields in the MR scanner. This has the advantage that it is compatible with Magnetic Resonance scanning apparatus.

Preferably the RF coil lies as close to the surface to be scanned as possible. For example it may be mounted to or in the vicinity of an outer surface of the housing. Preferably the housing and/or any fiducial marker unit does not substantially increase the distance between the RF coil and the surface being scanned.

As outlined above the housing encloses the RF coil. The outer surface of the housing may be shaped so as to define a recess that is externally accessible. For example it may include a well in which a fiducial can be placed.

As outlined above the at least one RF coil receives an RF signal. If required, the at least one RF coil may alternatively or additionally be configured to transmit an RF signal. In other words the at least one RF coil can be a receive coil, a transmit coil, or a transmit and receive coil.

The RF coil preferably is an antenna that is provided on either a flat or curved surface. The surface can be the housing itself, or it can be a separate substrate. The coil can have, for example, a circular, oval, rectangular, hexagonal or square cross-section, and has a region of maximum sensitivity. In a circular coil for example this region of maximum sensitivity may be found at the centre of the circle. Preferably the shape of the housing follows the shape of the coil.

Preferably the support is arranged so that the fiducial marker is located close to the centre of sensitivity of the RF coil; this allows the fiducial, when placed in the support, to be positioned to obtain maximum signal strength.

Advantageously, the container as mentioned above comprises a container body, for containing a fiducial marker. The container body may also have a first opening.

A container lid may be provided for sealing the first opening of the container. The container lid may comprise, for example, a bayonet, clip, screw or push fit cap. Conveniently, the container lid is shaped so as to eliminate, air pockets from the container and minimise susceptibility effects.

The container may conveniently be an appropriate size to fit into the recess of the RF coil apparatus, and may be retained in a fixed location in the recess as described above. Preferably the container is shaped so as to fit into the recess in the RF coil apparatus, so that the RF coil can lie close to the surface of the area of the subject being scanned as possible.

Conveniently, the container body is shaped so as to minimise susceptibility variation of the fiducial marker substance and its surrounding materials. For example, the thickness of the body of the container may not change sharply and/or the container may be located in a recess which has a similar shape and size as the outer surface of the container.

Advantageously, the RF coil apparatus comprises a plurality of RF coils. The RF coil apparatus may comprise a plurality of recesses, each recess receiving a fiducial marker, located within the RF field of the at least one RF coil. A plurality of fiducial marker units may also be provided if appropriate. This has the advantage that more than one fiducial marker can be used for one scan; also, more than one type of fiducial marker can be used for one scan.

The fiducial marker may comprise the substance which is being investigated during the scan. After performing the scan, the peak caused by the fiducial and the peak caused by that same substance in the body can be integrated. As the amount of fiducial substance present is known, the amount of the substance in the body can be then be calculated.

Alternatively, the fiducial substance may be a different substance to the substance being investigated. In this case the fiducial marker may give a signal peak in an area where there are no other signals, making the spectra more clear. Again, as the amount of fiducial is known, it is possible to integrate the peaks to calculate the amount of the substance being investigated present in an area of the body.

The apparatus may additionally comprise a cable for attachment to a magnetic resonance scanning machine.

A magnetic resonance scanning machine may also be provided that incorporates the above described RF coil apparatus. The magnetic resonance scanning apparatus may conveniently comprise an MRI machine or NMR apparatus.

According to a second aspect of the present invention, a method of using an apparatus for magnetic resonance spectroscopy is provided, the method comprising the steps of;
(i) taking an RF coil apparatus for magnetic resonance scanning comprising at least one RF coil and a housing for housing said at least one RF coil, the housing comprising a recess for receiving a fiducial marker, the recess being located within the RF field of said at least one RF coil;
(ii) accessing the recess; and
(iii) placing a fiducial in the recess;
characterised in that step (ii) can be performed without exposing said at least one RF coil.

Furthermore, apparatus for magnetic resonance spectroscopy is provided, the apparatus comprising:
at least one RF coil for receiving an RF signal; and
a housing for housing said at least one RF coil;
characterised in that a support is provided for locating a fiducial marker in a repeatable position relative to the housing, said repeatable position being located within the RF field of said at least one RF coil.

The invention is described above as apparatus for magnetic resonance spectroscopy. However, the apparatus may also be used for any type of magnetic resonance scanning, such as magnetic resonance imaging. Thus, a further aspect of the present invention provides RF coil apparatus for magnetic resonance scanning comprises at least one RF coil for receiving an RF signal, and a housing for housing said at least one RF coil; characterised in that the housing comprises a recess for receiving a fiducial marker, the recess being located within the RF field of said at least one RF coil, wherein a fiducial marker can be positioned in the recess without exposing the RF coil. The recess may be provided in the manner described above.

The invention will now be described, by way of example only, with reference to the accompanying drawings, in which:

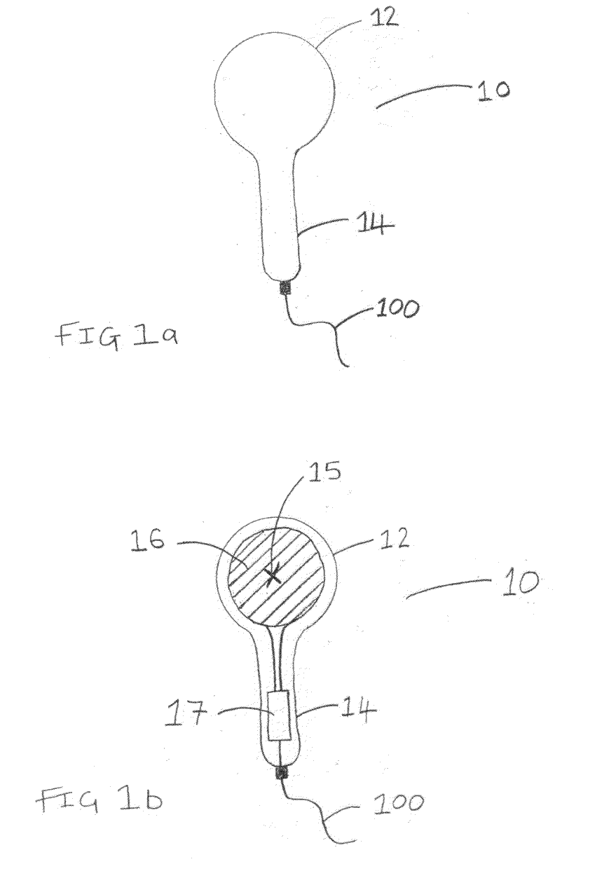

FIG. 1a shows a plan view of a prior art apparatus for magnetic resonance spectroscopy;

FIG. 1b shows a cross section through the plan view of a prior art magnetic resonance scanning apparatus as shown in FIG. 1a.

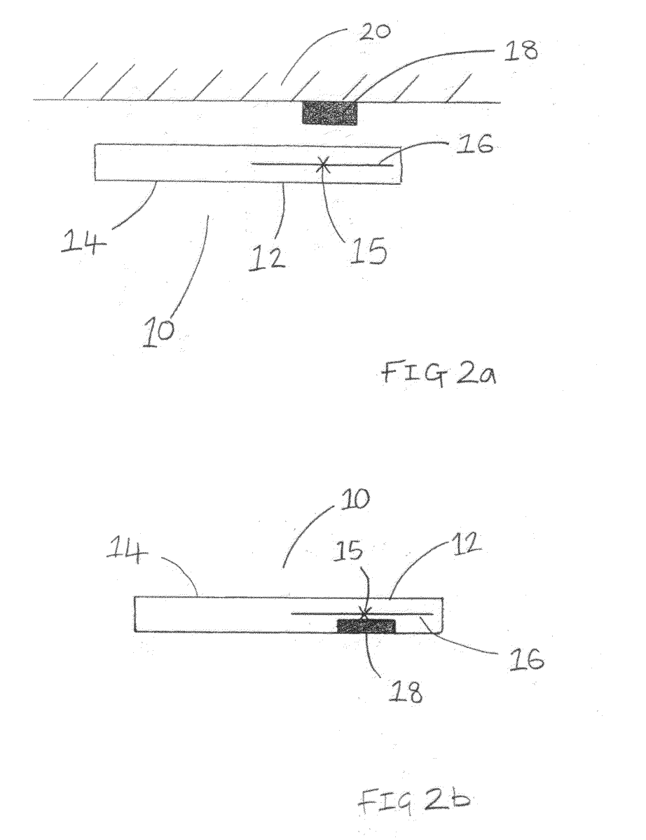

FIG. 2a shows a cross section through the side of a first prior art apparatus for magnetic resonance scanning;

FIG. 2b shows a cross section through the side of a second prior art apparatus for magnetic resonance scanning;

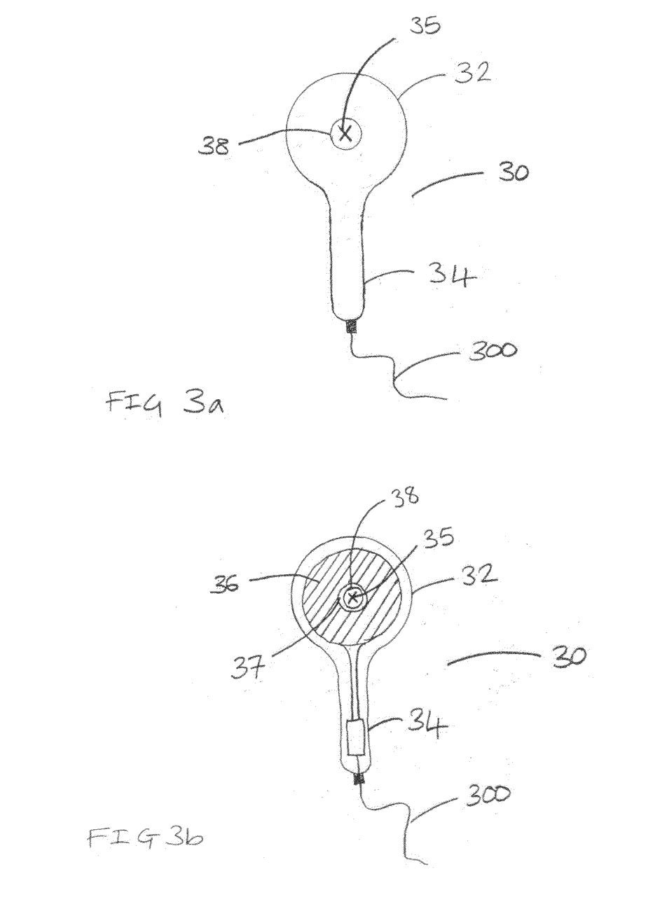

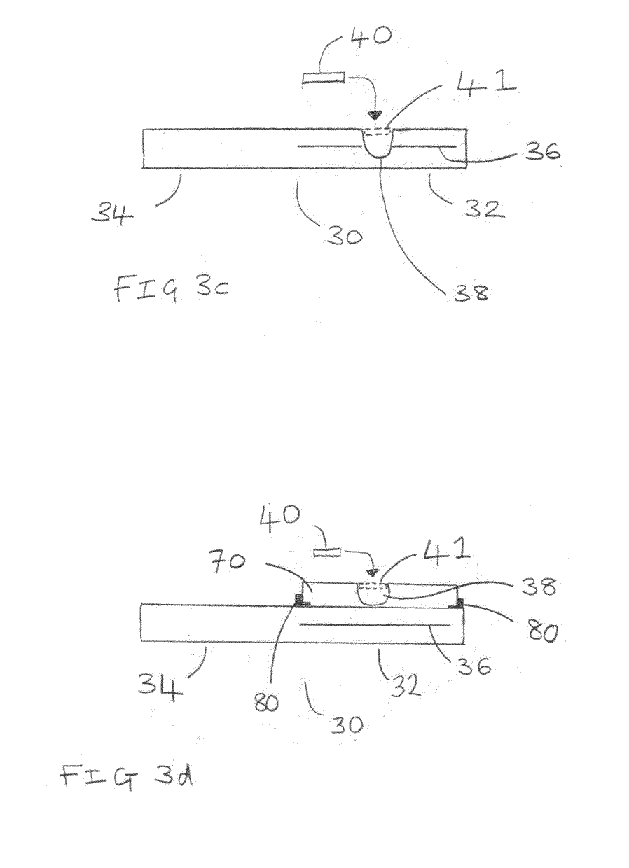

FIG. 3a shows a plan view of an apparatus for magnetic resonance scanning according to the present invention;

FIG. 3b shows a cross section through the plan view of an apparatus for magnetic resonance scanning as shown in FIG. 3a;

FIG. 3c shows a cross section through the side of an apparatus for magnetic resonance scanning according to the present invention;

FIG. 3d shows a cross section through the side of an alternative apparatus for magnetic resonance scanning according to the present invention;

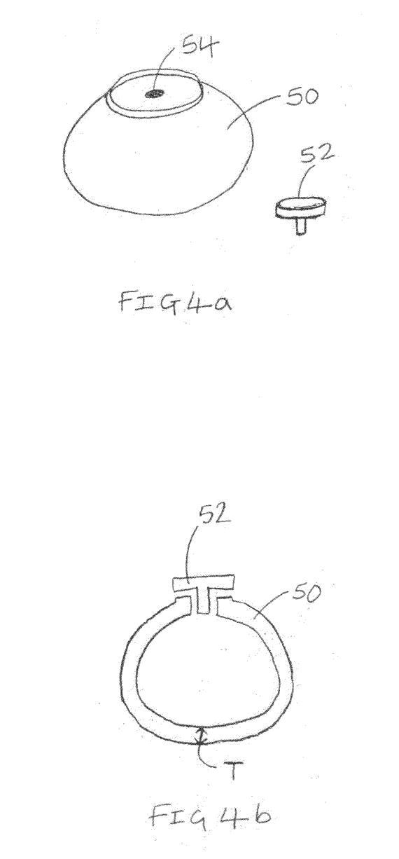

FIG. 4a shows a container for containing a fiducial marker, according to the present invention; and

FIG. 4b shows a cross section though a container for containing a fiducial marker, according to the present invention.

FIG. 1a shows a plan view of a prior art coil assembly for magnetic resonance spectroscopy apparatus. A housing 10 comprises two portions: a first portion 12, for positioning over the surface of the area to be scanned; and a second portion 14, for holding the apparatus whilst in use.

FIG. 1b shows a cross section through the plan view of a prior art magnetic resonance scanning apparatus as shown in FIG. 1a. The housing 10 houses an RF coil 16 for receiving an RF signal, and an electronics module 17 for interfacing the RF coil, via a cable 100, to a controller or a computer. The RF coil 16 has a region of maximum sensitivity 15 which lies centrally within coil 16, and thus centrally within the first portion 12 of the housing 10. The electronics module 17 is located within the second portion 14 of the housing 10.

FIG. 2a shows a cross section through a first prior art apparatus for magnetic resonance scanning. A glass vial containing a fiducial marker 18 is positioned between the surface 22 of the area to be scanned, and the housing 10 of the apparatus the glass vial containing the fiducial marker 18 is positioned above the maximum sensitivity region of the RF coil 15.

FIG. 2b shows a cross section through a second prior art apparatus for magnetic resonance scanning. Again the housing 10 houses an RF coil 16. In this apparatus, a glass vial containing a fiducial marker 18 is taped onto the inside of the housing 10, again above the maximum sensitivity region of the RF coil 15.

The prior art apparatus for magnetic resonance scanning has disadvantages as discussed previously. For example, the fiducial may be located a substantial distance from the maximum sensitivity region of the RF coil. Furthermore, the fiducial marker is not repeatably locatable and additionally is not externally accessible.

FIG. 3a shows a plan view of an apparatus for magnetic resonance scanning according to the present invention. The housing 30 comprises two portions, a first portion 32, for positioning over the surface of the area to be scanned; and a second portion 34, for holding the apparatus whilst in use. The first portion of the housing 32 comprises a recess 38, for receiving a fiducial marker. The recess is positioned as close as possible to the region of maximum sensitivity of the RF coil 35. A cable 300 is provided for electrically linking the RF coil to a magnetic resonance scanning machine.

FIG. 3b shows a cross section through the plan view of an apparatus for magnetic resonance scanning shown in FIG. 3a. FIG. 3c shows a cross section through the side of an apparatus for magnetic resonance scanning of FIGS. 3a and b. The RF coil 36 has a centrally located space 37 which is occupied by the recess 38. A fiducial marker may be placed in the recess 38, and the recess sealed by lid 40. Alternatively, a container containing a fiducial marker may be placed in the recess 38, and the recess sealed by lid 40. This allows the fiducial to be positioned as close as possible to the region of maximum sensitivity 35 of the RF coil 36. This gives the strongest signal from the fiducial marker, allowing greater accuracy in calculations of the quantity of fiducial present.

The recess 38 and its lid 40 are provided so that the fiducial, or fiducial container, does not protrude substantially from the housing 30 when the recess is sealed by the lid, as indicated by the dashed line 41. This allows the housing 30 and thus the RF coil 36 to lie closer to the surface to be scanned that the prior art as shown in FIG. 2a.

The lid 40 may be removed, and the fiducial marker changed, without exposing the RF coil. This makes the apparatus safer to use than the prior art apparatus as it does not expose the user to a high voltage electrical charge. Additionally, the apparatus enables the fiducial marker to be changed easily between scans, without having to break open the housing as would be required with the second prior art apparatus as shown in FIG. 2b.

FIG. 3d shows a cross section through the side of an alternative apparatus for magnetic resonance scanning according to the present invention. The housing is provided with a support comprising an attachment device, in this case a pair of clips 80 for receiving a fiducial marker unit 70. The fiducial marker unit 70 comprises a recess 38 for receiving a fiducial marker.

A fiducial marker may be placed in the recess 38, and the recess sealed by lid 40. Alternatively, a container containing a fiducial marker may be placed in the recess 38, and the recess sealed by lid 40. The recess 38 and its lid 40 are provided so that the fiducial, or fiducial container, does not protrude substantially from the unit 70 when the recess is sealed by the lid, as indicated by the dashed line 41.

FIG. 4a shows a container 50 for containing a fiducial marker, and a bung 52 for the container 50, according to the present invention. The container 50 has a hole 54 through which to fill it with the fiducial marker. The container has curved surfaces which do not vary sharply in thickness, so as to minimise susceptibility variations in the fiducial marker. Additionally the container 50 and bung 52 are shaped so as to eliminate air pockets from the container and thus minimise any interface of the fiducial with air.

FIG. 4b shows a cross section through a container 50 for containing a fiducial marker, according to the present invention. FIG. 4b shows that the thickness T of the container wall is substantially constant, this is to achieve minimal variations in the interactions of the fiducial particles with the air as discussed above.

The container has same shape as recess to help retain the container in a fixed location so that is doesn't move around during a scan. Also, the container and the recess having the same shape increases the separation between the fiducial and air and again help to minimise susceptibility variations.

1. Apparatus for magnetic resonance spectroscopy comprising:
at least one RF coil for receiving an RF signal; and
a housing for housing said at least one RF coil;
characterised in that a support is provided for locating a fiducial marker in a repeatable position relative to the housing, the fiducial marker providing a reference peak in a magnetic resonance spectroscopy spectrum, wherein said repeatable position is located within the RF field of said at least one RF coil.

2. An apparatus according to claim 1 wherein the support enables a fiducial marker to be located in said repeatable position without exposing the RF coil.

3. An apparatus according to claim 1 wherein the support comprises an attachment device for releasably retaining a fiducial marker unit.

4. An apparatus according to claim 3 wherein the attachment device comprises a clip.

5. An apparatus according to claim 3 comprising a fiducial marker unit, wherein the fiducial marker unit comprises a recess for receiving a fiducial marker.

6. An apparatus according to claim 1 wherein the housing comprises a recess that provides said support, the recess being suitable for receiving a fiducial marker.

7. An apparatus according to claim 6 wherein the outer surface of the housing is shaped so as to define the recess, wherein the recess is externally accessible.

8. An apparatus according to claim 5 wherein the RF coil has a region of maximum sensitivity and the recess is provided close to said region of maximum sensitivity.

9. An apparatus according to claim 5 wherein the recess itself contains the fiducial substance.

10. An apparatus according to claim 5 wherein the recess is shaped to receive a container containing a fiducial substance.

11. An apparatus according to claim 10 wherein the recess comprises a through hole into which the container fits.

12. An apparatus according to claim 10, comprising a container located in the recess, the container containing a fiducial substance.

13. An apparatus according to claim 12 wherein the container comprises a container body having a first opening.

14. An apparatus according to claim 13 wherein a container lid is provided for sealing the first opening.

15. An apparatus according to claim 14 wherein the container lid comprises at least one of a bayonet, clip, screw or push fit cap.

16. An apparatus according to claim 14 wherein the container lid is shaped so as to eliminate air pockets from the container thereby minimising susceptibility effects.

17. Apparatus according to claim 13 wherein the container body is shaped so as to minimise susceptibility variation of the fiducial marker substance and its surrounding materials.

18. An apparatus according to claim 5 wherein the recess has a substantially smooth profile.

19. An apparatus according to claim 5 wherein a lid is provided to seal the recess.

20. An apparatus according to claim 19 wherein the lid does not protrude substantially from the RF coil apparatus.

21. An apparatus according to claim 5 comprising a plurality of recesses for receiving fiducial markers, wherein each recess is located within the RF field of the at least one RF coil.

22. An apparatus according to claim 1 comprising a plurality of RF coils.

23. An apparatus according to claim 1 wherein the housing and support consist of materials that have substantially no effect on the RF or magnetic fields in the MR scanner.

24. An apparatus according to claim 23 wherein said materials comprise plastic.

25. An apparatus according to claim 1 wherein the housing does not substantially increase the distance between the at least one RF coil and the surface being scanned.

26. An apparatus according to claim 1 wherein the at least one RF coil is configured to transmit an RF signal.

27. An apparatus according to claim 1 wherein the RF coil is provided on at least one of a flat and curved surface.

28. An apparatus according to claim 1 comprising a cable for electrically linking the at least one RF coil to a magnetic resonance scanning machine.

29. A magnetic resonance scanning machine comprising apparatus according to claim 1.

30. A machine according to claim 29 comprising magnetic resonance imaging (MRI) apparatus.

31. A method of using an apparatus for magnetic resonance spectroscopy comprising the steps of;
(i) taking an apparatus for magnetic resonance scanning comprising at least one RF coil and a housing for housing said at least one RF coil, the housing comprising a recess for receiving a fiducial marker, the recess being located within the RF field of said at least one RF coil;
(ii) accessing the recess; and
(iii) placing a fiducial in the recess;
wherein step (ii) can be performed without exposing said at least one RF coil.

32. Apparatus for magnetic resonance scanning comprising:
at least one RF coil for receiving an RF signal; and
a housing for housing said at least one RF coil;
wherein the housing comprises a recess for receiving a fiducial marker, the recess being located within the RF field of said at least one RF coil, wherein a fiducial marker can be positioned in the recess without exposing the RF coil.

33. An apparatus according to claim 32 wherein the recess is cooperable with a fiducial marker so as to provide a repeatable position of said fiducial marker with respect to the housing.

34-35. (canceled)
